(19) United States
(12) Patent Application Publication
(10) Pub. No.: US 2005/0147962 A1
(43) Pub. Date: Jul. 7, 2005

(54) Display of dimeric proteins on phage

(76) Inventors: Christopher R. Wagstrom, Los Altos, CA (US); David S. Wilson, Mountain View, CA (US); Steffen Nock, Redwood City, CA (US)

Correspondence Address: PERKINS COIE LLP, P.O. BOX 2168, MENLO PARK, CA, 94026, US

(21) Appl. No.: 10/717,735

(22) Filed: Nov. 19, 2003

Related U.S. Application Data
(60) Provisional application No. 60/427,736, filed on Nov. 19, 2002.

(51) Int. Cl. C12Q 1/70; C07H 21/04; C12N 1/21; C12N 15/74; C07K 16/18

(52) U.S. Cl. 435/5; 435/69.1; 435/252.3; 435/472; 530/388.1; 536/23.53

(57) ABSTRACT

Expression vectors for expressing multimeric polypeptides that are anchored on surfaces of genetically replicable packages are disclosed. The expression vectors include a vector segment encoding a polypeptide sequence having three polypeptide segments. One of the segments contains a cleavable peptide sequence cleavable by a proteolytic agent, and another segment has an anchoring peptide sequence for anchoring the multimeric polypeptide to the surface of the genetically replicable package. The cleavable peptide sequence is cleaved by the proteolytic agent and the first segment associates with the third segment to form the multimeric polypeptide. Also disclosed are methods, host cells, and kits employing the expression vectors.

The present application relates to methods and compositions for expressing multimeric polypeptides, such as antibody fragments, anchored onto a surface of a genetically replicable package, preferably bacteriphage.

### FIELD OF THE INVENTION

The present invention relates to methods and compositions for expressing multimeric polypeptides, such as antibody fragments, anchored onto a surface of a genetically replicable package, preferably bacteriophage.

### BACKGROUND OF THE INVENTION

There has been considerable interest in the production of antibody fragments and analogous entities in recent years (Hudson, P J and Souriau, C (2001) Expert Opin. Biol. Ther. 1(5):845-55). One fragment of particular interest is the Fab fragment, which consists of a light chain comprising a variable and a constant domain (V_{L}-C_{L}) bound to a heavy chain comprising a variable and constant domain (V_{H}-CH1). The intermolecular forces, consisting of numerous non-covalent interactions and one disulfide bond, bring about the association of these domains in whole antibodies and also in Fab fragments. Because properly folded Fab fragments contain disulfide bonds, Fabs generally must be expressed in an oxidizing environment. In bacteria, the periplasm is such an environment, so the Fab polypeptides need to contain secretion leader sequences that cause them to be translocated into the periplasm, where proper folding occurs.

Fusion phage are filamentous bacteriophage vectors that include foreign peptides and proteins cloned into a phage coat gene and displayed as part of a phage coat protein. Phage display is a powerful technique for identifying peptides or proteins that bind to other molecules. In this method, a DNA coding region is inserted into the bacteriophage genome such that the expressed peptide or protein is displayed on the surface of the phage particle as a fusion to an endogenous protein. Simple panning procedures enable phage encoding desirable molecules to be selected from large libraries of recombinants. Phage display has been used to identify peptides that bind to receptors, substrates, or inhibitors of enzymes, epitopes, improved antibodies, altered enzymes, and cDNA clones (Yip, Y. L. and Ward, R. L. (2002) Curr. Pharm. Biotechnol. 3(1):2943).

The commonly used coat genes for the production of fusion phage are the pVIII gene and the pIII gene. Approximately 3900 copies of pVIII make up the major portion of the tubular virion protein coat. Each pVIII coat protein lies at a shallow angle to the long axis of the virion, with its C-terminus buried in the interior close to the DNA and its N-terminus exposed to the external environment. Five copies of the pill coat protein are located at the terminal end of each virion. Insertion of polypeptide segments into the coat protein genes allows the production of phage displayed polypeptide libraries. A typical display library contains 10 to 1000 copies of as many as 10¹¹ different-sequence polypeptides. Thus, phage display is useful for screening large numbers of polypeptides for molecules of interest with desired binding characteristics.

Fab fragments displayed on filamentous phage are typically produced by separately expressing the heavy and light chains. Each chain contains a secretion leader sequence, which causes it to be translocated to the periplasm. After translocation, the leader sequences are cleaved off by a signal peptidase. Then, the heavy and light chains can associate to form the Fab fragments. This co-expression can be performed by having the chains expressed from a single phage/phagemid vector, or by expressing the chains on separate vectors with either the heavy or light chain being expressed from the phage/phagmid vector and the other chain being expressed from a plasmid vector. The main problem with this method is the non-stoichiometric expression and/or translocation of the heavy and light chains from the cells, thereby wasting cellular metabolism in unproductive synthesis. Moreover, it is generally thought that the expression of the heavy chain without the light chain is often harmful to the cells that express it, making it difficult to obtain concentrations suitable for industrial production.

These difficulties may be avoided by producing a single polypeptide containing a single secretion leader sequence, a light chain variable region and a heavy chain variable region, and a linking peptide sequence which joins the two variable regions together. This linking peptide sequence is designed so that after the single polypeptide has been expressed, the two domains can associate together to form a molecule analogous to an Fab fragment, except that only the variable regions are present. These molecules, referred to as single-chain variable fragments (scFv), have a molecular weight of about half of that of Fab fragments, since they lack the CH1 domain from the heavy chain and the CL domain from the light chain. Genetic constructs encoding scFv's have some clear advantages in the production of antibody fragments. First, the two domains are produced in equal quantities. Second, the two domains are produced at high local concentration and therefore association is strongly favored. However, the resulting scFv's are disappointing in their performance when compared to Fab fragments. The main reasons for this is that the Fv fragments lack the constant regions (CH1 and CL) that provide most of stabilizing interactions between the heavy and light chain, including a disulfide bond between CH1 on the heavy chain and CL on the light chain.

Thus, it would be desirable to express the associative portions of two peptide segments, e.g., a heavy chain and a light chain, as parts of a single polypeptide in which they are connected through a linking peptide sequence. However, this connection should incorporate a site for cleavage by an enzyme produced by the transformed organism that is expressing the polypeptide. After or during expression of the single polypeptide it is cut at the cleavage site while still within the culture where it has been expressed, thereby detaching the portions of the peptide segments from each other and allowing them to associate spontaneously together. Thus, the two domains would be produced and translocated into the periplasm in equal quantities, and they would have the stabilizing interactions between the constant domains of the heavy and light chains. The present invention is designed to meet these needs.

### SUMMARY OF THE INVENTION

The invention includes, in one aspect, an expression vector for expressing a multimeric polypeptide anchored on a surface of a genetically replicable package formed by a host. The expression vector includes a vector segment encoding a polypeptide sequence. The polypeptide sequence has a first polypeptide segment, a second polypeptide segment having therein a cleavable peptide sequence cleavable by a proteolytic agent, and a third polypeptide segment having therein an anchoring peptide sequence for anchoring the multimeric polypeptide to the surface of the genetically replicable package. The second polypeptide segment is between the first polypeptide segment and the third segment. The cleavable peptide sequence is cleaved by the proteolytic agent and the first segment associates with the third segment to form the multimeric polypeptide.

In one embodiment of the invention, the first and third polypeptide segments include an amino acid sequence derived from antibody light and heavy chains. In another embodiment, the first and third polypeptide segments include the antigen binding regions of the variable domains of antibody light and heavy chains.

In another embodiment of the invention the first polypeptide segment includes the variable domain and the constant domain of an antibody light chain, and the third polypeptide segment includes the variable domain and a constant domain of the antibody heavy chain, such that when the first and third segments associate, the product is a Fab antibody fragment. In yet another embodiment, the first polypeptide segment includes the variable domain and the CH1 domain of an antibody heavy chain, and the third polypeptide segment comprises the variable domain and the constant domain of the antibody light chain, such that when the first and third segments associate, the product is a Fab antibody fragment. Alternatively, the first polypeptide segment includes the variable domain and the constant domain of the antibody light chain, and the third polypeptide segment includes the variable domain and the CH1 domain of an antibody heavy chain, such that when the first and third segments associate, the product is a Fab antibody fragment.

When the first and third polypeptide segments include the variable domains of the light and heavy chains, of a single antibody, they may associate to form an Fv antibody fragment.

In one embodiment, the first polypeptide segment is N-terminal to-the second polypeptide segment, the second polypeptide segment is N-terminal to the third polypeptide segment, and the vector segment encoding the third polypeptide segment further includes one or more suppressable nonsense codon(s) N-terminal to the anchoring segment.

The third polypeptide segment may further include a cleavable peptide sequence cleavable by a second proteolytic agent. In one embodiment, the first and second proteolytic agents are identical. Alternatively, the first and second proteolytic agents are different.

The proteolytic agent may be a chemical proteolytic agent or an enzymatic proteolytic agent. The chemical proteolytic agent may be an acid. In one embodiment of the invention, the proteolytic agent is expressed by the host. In another embodiment, the proteolytic agent is added such that it contacts and cleaves the second polypeptide segment.

In one embodiment, the cleavable peptide sequence includes the sequence represented by SEQ ID NO:1. In another embodiment, the cleavable peptide sequence is not found in either the first or third polypeptide segments, and is recognized as a protein cleavage site by a proteolytic agent encountered in the host.

The polypeptide sequence may further include one or more leader sequence(s) positioned upstream of the first polypeptide segment or third polypeptide segment or both first and third polypeptide segments.

The anchoring peptide may include a segment encoding a phage coat protein.

The phage coat protein may be selected from the group consisting of plasmids, phages, cosmids, phagemids, and viral vectors. The expression vector may be selected from the group consisting of M13, f1, fd, If1, Ike, Xf, Pf1, Pf3, λ, T4, T7, P2, P4, φX-174, MS2 and f2.

The genetically replicable package is selected from the group consisting of a bacteriophage, a virus, a cell and a spore.

In one embodiment, the cell is a bacterial cell. The bacterial cell may be selected from the group consisting of strains of Escherichia coli, Salmonella typhimurium, Pseudomonas aeruginosa, Klebsiella pneumonial, Neisseria gonorrhoeae, and Bacillus subtilis. In another embodiment, the cell is a yeast cell.

In yet another embodiment, the genetically replicable package is a filamentous bacteriophage specific for Escherichia coli and the anchoring peptide is a phage coat protein selected from the group consisting of coat protein III, coat protein pVI and coat protein VIII. The filamentous bacteriophage may be M13 or fd.

In one embodiment, the proteolytic agent is encoded by a nucleic acid sequence in the expression vector. Alternatively, the proteolytic agent is encoded by a nucleic acid sequence in a second expression vector.

The cleavable peptide sequence includes, in one embodiment, a disordered region cleavable by the proteolytic agent. Alternatively, the cleavable peptide sequence includes a specific peptide cleavage site cleavable by the proteolytic agent. In a related embodiment, the cleavable peptide sequence includes a cleavage site for urokinase, pro-urokinase, thrombin, enterokinase, plasmin, plasminogen, TGF-β, staphylokinase, thrombin, Factor IXa, Factor Xa, a metalloproteinase, an interstitial collagenase, a gelatinase or a stromelysin. In yet another embodiment, the cleavable peptide sequence is cleavable by a protease selected from the group consisting of degP, degQ, degS and tsp.

The cleavable peptide sequence may include a self-cleaving domain. The self-cleaving domain may be derived from an intein.

In another aspect, the invention includes a host cell including the expression vector described above. The proteolytic agent may be a native proteolytic agent. In one embodiment, the proteolytic agent is localized in the periplasm. In another embodiment, the proteolytic agent is localized in the cytoplasm.

The invention also includes, in yet another aspect, a method of producing a multi-subunit protein. The method includes transforming a host cell with an expression vector described above, and displaying the multi-subunit protein encoded by the vector onto the surface of the genetically replicable package.

In one embodiment, the expression vector includes nucleotide sequences encoding functional portions of heterodimeric receptors selected from the group consisting of antibodies, T cell receptors, integrins, hormone receptors and transmitter receptors.

In yet, still another aspect of the invention, a library of antibodies or antibody fragments is made. In one embodiment, a library of bacteriophage or phagemids, each carrying on its outer surface, one of a plurality of different-sequence polypeptides is provided. The different-sequence polypeptides include one of a plurality of first different-sequence heterologous polypeptide segments, one of a plurality of a second different-sequence heterologous polypeptide segments, and joining the two segments, a peptide linker that has a cleavable peptide sequence that is not found in either of said polypeptide segments, and is recognized as a protein cleavage site by a proteolytic enzyme encountered in a bacteriophage host during bacteriophage biogenesis. Cleavage of the linker by the host proteolytic enzyme results in a multimeric protein on the surface of a bacteriophage. Each protein has a plurality of different-sequence first and second polypeptides, and a protein activity related to the sequences of the first and second polypeptides.

The protein activity may be a specific binding affinity for a selected molecule of interest.

In another embodiment, the invention includes a library of bacteriophage genomes or phagemids. In this embodiment, each genome encodes one of a plurality of first different-sequence heterologous polypeptide segments, one of a plurality of a second different-sequence heterologous polypeptide segments, and joining the two segments, a peptide linker that has a cleavable peptide sequence that is not found in either of said polypeptide segments, and is recognized as a protein cleavage site by a proteolytic enzyme encountered in a bacteriophage host during bacteriophage biogenesis. Cleavage of the linker by the host proteolytic enzyme results in a multimeric protein on the surface of a bacteriophage, each protein (i) having a plurality of different-sequence first and second polypeptides, and (ii) a protein activity related to the sequences of the first and second polypeptides.

In yet another aspect of the invention, a method of identifying one or more multimeric proteins having a desired above-threshold activity is provided. The method includes producing a library a bacteriophage or phagemids, each carrying on its outer surface, one of a plurality of different-sequence polypeptides. The different-sequence polypeptides include one of a plurality of first different-sequence heterologous polypeptide segments, one of a plurality of a second different-sequence heterologous polypeptide segments, and joining the two segments, a peptide linker that has a cleavable peptide sequence that is not found in either of said polypeptide segments, and is recognized as a protein cleavage site by a proteolytic enzyme encountered in a bacteriophage host during bacteriophage biogenesis. Cleavage of the linker by the host proteolytic enzyme results in a multimemric protein on the surface of a bacteriophage, each protein (i) having a plurality of different-sequence first and second polypeptides, and (ii) a protein activity related to the sequences of the first and second polypeptides. Bacteriophage in the library that have the above-threshold activity are identified.

In one embodiment, the method further includes sequencing the portion of the genome(s) of the identified bacteriophage that encode said first and second polypeptides.

In another embodiment, the invention provides a method for creating a library of antibodies or antibody fragments. The method includes obtaining a biological sample, introducing the biological sample to a cell population capable of producing antibodies, reverse transcribing the light chain region and heavy chain region mRNA, or fragments thereof, of the cell population, amplifying and linking the two antibody fragment cDNA sequences with a linker comprising a nucleic acid sequence which encodes an amino acid sequence capable of being cleaved by a proteolytic agent, amplifying the linked sequences to create a population of DNA fragments which encode the two antibody fragments, cloning the population of DNA fragments into expression vectors and amplifying the cloned expression vectors, and selecting a subpopulation of expression vectors which encode antibodies or antibody fragments directed against the biological sample and amplifying the subpopulation selected to produce the library of antibodies or antibody fragments.

In one embodiment, the amplifying is performed by PCR.

In yet another embodiment of the invention, a method for creating a patient-specific library of antibodies is provided. The method includes obtaining a sample of tissue from a patient, introducing the sample to a cell population capable of producing antibodies, reverse transcribing the light chain region and heavy chain region mRNA, or fragments thereof, of the cell population, amplifying and linking the two antibody fragment cDNA sequences with a linker comprising an amino acid sequence capable of being cleaved by a proteolytic agent, amplifying the linked sequences to create a population of DNA fragments which encode the two antibody fragments, cloning the population of DNA fragments into expression vectors and selecting a subpopulation of expression vectors which encode recombinant anti-sample antibody fragments, cloning the subpopulation of DNA fragments selected in-frame into expression vectors which encode antibody constant regions to produce intact antibody genes; and expressing the subpopulation of intact antibody genes to produce the library of patient-specific antibodies.

Another aspect of the invention provides an expression vector for expressing a multimeric polypeptide anchored on a surface of a genetically replicable package formed by a host. The expression vector includes a vector segment encoding a polypeptide sequence. The polypeptide sequence has a first polypeptide segment having therein a first variable domain and a first constant domain of an antibody, a second polypeptide segment, and a third polypeptide segment having therein (a) a second variable domain and a second constant domain of an antibody, and (b) an anchoring peptide sequence for anchoring said multimeric polypeptide to said surface of said genetically replicable package. The second polypeptide segment is between the first polypeptide segment and the third segment and has a length that prohibits the first and third polypeptide segments from associating intramolecularly to form a single-chain Fab, but allows two copies of the polypeptide to associate intermolecularly to form a di-Fab.

In one embodiment, the second polypeptide segment further comprises a cleavable peptide sequence cleavable by a proteolytic agent.

In another embodiment, the first polypeptide segment is N-terminal to the second polypeptide segment, the second polypeptide segment is N-terminal to the third polypeptide segment, and the vector segment encoding the third polypeptide segment further includes one or more suppressable nonsense codon(s) N-terminal to the anchoring segment.

The third polypeptide segment may further include a cleavable peptide sequence cleavable by a proteolytic agent.

The proteolytic agents described above may be chemical proteolytic agents or enzymatic proteolytic agents.

In one embodiment, the proteolytic agent is expressed by the host. Alternatively, the proteolytic agent is added such that it contacts and cleaves the second polypeptide segment.

The chemical proteolytic agent may be an acid.

The cleavable peptide sequence may include the sequence represented by SEQ ID NO:1.

In one embodiment, the cleavable peptide sequence is not found in either the first or third polypeptide segments, and is recognized as a protein cleavage site by a proteolytic agent encountered in the host.

In another embodiment of the invention, the polypeptide sequence further includes one or more leader sequence(s) positioned upstream of the first polypeptide segment or third polypeptide segment or both first and third polypeptide segments.

The anchoring peptide may include a segment encoding a phage coat protein.

The expression vector may be selected from the group consisting of plasmids, phages, cosmids, phagemids, and viral vectors. In a related embodiment, the expression vector is selected from the group consisting of M13, f1, fd, If1, Ike, Xf, Pf1, Pf3, λ, T4, T7, P2, P4, φX-174, MS2 and f2.

The genetically replicable package may be selected from the group consisting of a bacteriophage, a virus, a cell and a spore.

In one embodiment, the cell is a bacterial cell. The bacterial cell may be selected from the group consisting of strains of Escherichia coli, Salmonella typhimurium, Pseudomonas aeruginosa, Klebsiella pneumonial, Neisseria gonorrhoeae, and Bacillus subtilis.

In another embodiment, the cell is a yeast cell.

In yet another embodiment, the genetically replicable package is a filamentous bacteriophage specific for Escherichia coli and the anchoring peptide is a phage coat protein selected from the group consisting of coat protein III, coat protein pVI and coat protein VIII.

In yet, still another embodiment, the filamentous bacteriophage is M13 or fd.

The proteolytic agent may be encoded by a nucleic acid sequence in the expression vector. Alternatively, the proteolytic agent is encoded by a nucleic acid sequence in a second expression vector.

In one embodiment, the cleavable peptide sequence includes a disordered region cleavable by the proteolytic agent. In another embodiment, the cleavable peptide sequence includes a specific peptide cleavage site cleavable by the proteolytic agent.

The cleavable peptide sequence may include a cleavage site for urokinase, pro-urokinase, thrombin, enterokinase, plasmin, plasminogen, TGF-β, staphylokinase, thrombin, Factor IXa, Factor Xa, a metalloproteinase, an interstitial collagenase, a gelatinase or a stromelysin.

In one embodiment of the invention, the cleavable peptide sequence is cleavable by a protease selected from the group consisting of degP, degQ, degS and tsp.

The cleavable peptide sequence may include a self-cleaving domain. The self-cleaving domain may be derived from an intein.

Also disclosed is a host cell comprising the expression vector described above.

Another aspect of the invention includes a method of producing a multi-subunit protein. The method includes transforming a host cell with the expression vector described above, and displaying the multi-subunit protein encoded by the vector onto the surface of the genetically replicable package.

Yet another aspect of the invention includes a library of antibodies or antibody fragments made according to the method described above.

Also disclosed is a method of producing a di-Fab. The method includes expressing the polypeptide sequence from any of the expression vectors described above under conditions effective to allow the two copies of the polypeptide to associate intermolecularly to form a di-Fab.

These and other objects and features of the invention will be more fully appreciated when the following detailed description of the invention is read in conjunction with the accompanying drawings.

### BRIEF DESCRIPTION OF THE DRAWINGS

FIG. 1 schematically illustrates construction of polypeptides encoded by the expression vectors and libraries according to one embodiment of the invention where two polypeptide segments are joined together by a cleavable linker, and fused to an anchoring peptide;

FIG. 2 depicts a portion of the polypeptide encoded by an expression vector that includes a leader sequence at the amino terminus of the two polypeptide fragments for secretion according to another embodiment of the invention;

FIGS. 3A-3B illustrate the linear sequence of the fusion protein encoded by the fusion gene having a flexible linker, which may be cleavable (FIG. 3B), according to other embodiments of the invention;

FIGS. 4A-4B show embodiments of the sequence illustrated in FIGS. 3A-3B, with a relatively short linker, which may be cleavable (FIG. 4B), that allows a polypeptide dimer to be processed and folded according to yet another embodiment of the invention.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al. (2001) “Molecular Cloning: A Laboratory Manual” Cold Spring Harbor Press, 3rd Ed.; and Ausubel, F. M., et al. (1993) in Current Protocols in Molecular Biology, for definitions and terms of the art. It is to be understood that this invention is not limited to the particular methodology, protocols, and reagents described, as these may vary.

The terms “protein,” “polypeptide,” or “peptide” as used herein refers to a biopolymer composed of amino acid or amino acid analog subunits, typically some or all of the 20 common L-amino acids found in biological proteins, linked by peptide intersubunit linkages, or other intersubunit linkages. The protein has a primary structure represented by its subunit sequence, and may have secondary helical or pleat structures, as well as overall three-dimensional structure. Although “protein” commonly refers to a relatively large polypeptide, e.g., containing 100 or more amino acids, and “peptide” to smaller polypeptides, the terms are used interchangeably herein. That is, the term protein may refer to a larger polypeptide, as well as to a smaller peptide, and vice versa.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Several different regions of an antibody contain conserved sequences. Extensive amino acid and nucleic acid sequence data displaying exemplary conserved sequences is compiled for immunoglobulin molecules by Kabat et al., in Sequences of Proteins of Immunological Interest, National Institutes of Health, Bethesda, Md., 1987.

The term “antibody fragment” refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′) (2), scFv, Fv, dsFv diabody, and Fc fragments. The antibody fragment can optionally be a single chain antibody fragment. Alternatively, the fragment can comprise multiple chains which are linked together, for instance, by disulfide linkages. The fragment can also optionally be a multimolecular complex. A functional antibody fragment will typically comprise at least about 50 amino acids and more typically will comprise at least about 200 amino acids.

A typical antibody structural unit is known to comprise a tetramer. Each tetramer is composed of two identical pairs of polypeptide chains, each pair having one “light”) (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The variable region of the heavy or light chain typically comprises four framework regions each containing relatively lower degrees of variablity that includes lengths of conserved sequences. Framework regions are typically conserved across several or all immunoglobulin types and thus conserved sequences contained therein are particularly suited for preparing repertoires having several immunoglobulin types.

The term “above threshold” refers to a level of a protein activity or protein binding that is greater than the level of the activity observed with normal activity or nonspecific binding. For some proteins, no or infinitesimally low levels of activity or binding may be present. For other proteins, detectable activities may be present normally. Thus, the term further contemplates a level that is significantly above the level found typically. The term “significantly” refers to statistical significance, and generally means at least a two-fold greater level of activity is present. However, a significant difference between levels of activities depends on the sensitivity of the assay employed, and must be taken into account for each activity or binding assay.

The term “nucleic acid sequence” includes RNA, DNA and cDNA molecules. It will be understood that, as a result of the degeneracy of the genetic code, a multitude of nucleotide sequences encoding given peptides such as antibody fragments may be produced. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyl-2-thiouracil, 5-carboxymethylaminomethyluracil, dihydrouracil, inosine, N6-isopentenyladenine, 1-methyladenine, 1methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “heterologous” as it relates to nucleic acid sequences such as coding sequences and control sequences, denotes sequences that are not normally associated with a region of a vector or replicable genetic package, and/or are not normally associated with a particular host cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

The term “isolated” when used in relation to a nucleic acid or protein sequence refers to a sequence that is identified and separated from at least one contaminant with which it is typically associated in its natural source. Isolated nucleic acid or protein is present in a form or setting that is different from that in which it is found in nature. In contrast, non-isolated nucleic acids and proteins are in the state in which they exist in nature.

The term “purified” or “purify” refers to the removal of contaminants from a sample.

As used herein, “coding sequence” or a sequence which “encodes” a particular polypeptide, is a nucleic acid sequence which is transcribed (in the case of DNA) and translated (in the case of mRNA) into a polypeptide in vitro or in vivo, when placed under the control of appropriate regulatory sequences. The boundaries of the coding sequence are determined by a start codon at the 5′ (amino) terminus and a translation stop codon at the 3′ (carboxy) terminus. A coding sequence may include, but is not limited to, cDNA from prokaryotic or eukaryotic mRNA, genomic DNA sequences from prokaryotic or eukaryotic DNA, and synthetic DNA sequences. A transcription termination sequence will typically be located 3′ to the coding sequence.

The phrase “specifically binds to a protein” or “specifically immunoreactive with”, when referring to an antibody refers to a binding reaction which is determinative of the presence of the protein in the presence of a heterogeneous population of proteins and other biomolecules. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies: A Laboratory Manual, Cold Spring Harbor Publications, New York, for descriptions of immunoassay formats and conditions that may be used to determine specific immunoreactivity.

The term “conservative substitution” is used in reference to proteins or peptides to reflect amino acid substitutions that do not substantially alter the activity (specificity or binding affinity) of the molecule. Typically, conservative amino acid substitutions involve substitution of one amino acid for another amino acid with similar chemical properties (e.g., charge or hydrophobicity). The following six groups each contain amino acids that are typical conservative substitutions for one another:
i. Alanine (A), Serine (S), Threonine (T);
ii. Aspartic acid (D), Glutamic acid (E);
iii. Asparagine (N), Glutamine (Q);
iv. Arginine (R), Lysine (K);
v. Isoleucine (I), Leucine (L), Methionine (M), Valine (V); and
vi. Phenylalanine (F), Tyrosine (Y), Tryptophan (W).

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “wild-type” refers to a gene or gene product which has the characteristics of that gene or gene product when isolated from a naturally occurring source. A wild-type gene is that which is most frequently observed in a population and is thus arbitrarily designated the normal or wild-type form of the gene. In contrast, the term “modified” or “mutant” referes to a gene or gene product which displays modifications in sequence and/or functional properties, i.e., altered characteristics, when compared to the wild-type gene or gene product.

As used herein, the term “Vector” refers to a nucleic acid construct designed for transfer between different host cells. A vector may have the ability to incorporate and express heterologous DNA fragments in a foreign host. Many prokaryotic and eukaryotic expression vectors are commercially available. Selection of appropriate expression vectors is within the knowledge of those having skill in the art. A vector may be generated recombinantly or synthetically, with a series of specified nucleic acid elements that permit transcription of a particular nucleic acid in a host or in vitro. Vector segments can be incorporated into a plasmid, chromosome, mitochondrial DNA, plastid DNA, virus, or nucleic acid fragment. Typically, the vector includes, among other sequences, a nucleic acid sequence to be transcribed and a promoter.

As used herein, the term “selectable marker-encoding nucleotide sequence” refers to a nucleotide sequence which is capable of expression in host cells and where expression of the selectable marker confers to cells containing the expressed gene the ability to grow in the presence of a corresponding selective agent.

As used herein, the terms “promoter” and “transcription initiator” refer to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”, as defined below) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

As used herein, the term “expression” refers to the process by which a polypeptide is produced based on the nucleic acid sequence of a gene. The process includes both transcription and translation.

The term “signal sequence” refers to a sequence of amino acids at the N-terminal portion of a protein which facilitates the secretion of the mature form of the protein outside the cell. The mature form of the extracellular protein may lack the signal sequence if it is cleaved off during the secretion process.

The term “amplifying” refers to repeated copying of a specified sequence of nucleotides resulting in an increase in the amount of the specified sequence of nucleotides.

The term “PCR” refers to the polymerase chain reaction that is the subject of U.S. Pat. Nos. 4,683,195 and 4,683,202 to Mullis, as well as other improvements now known in the art.

The term “sequencing” refers to a procedure for determining the order in which nucleotides occur in a protein or nucleotide sequence.

By the term “host cell” is meant a cell that contains a vector and supports the replication, or transcription and translation (expression) of the expression construct. Host cells for use in the present invention can be prokaryotic cells, such as E. coli, or eukaryotic cells such as yeast, plant, insect, amphibian, or mammalian cells.

All publications and patents cited herein are expressly incorporated herein by reference for the purpose of describing and disclosing compositions and methodologies which might be used in connection with the invention.

II. Method of the Invention

One aspect of the invention includes a method for making a multimeric polypeptide anchored onto a surface of a genetically replicable package. A vector is used to encode the multimeric polypeptide. The multimeric polypeptide includes at least three segments: (i) a first polypeptide segment that has an anchoring peptide therein for anchoring the multimeric polypeptide to the surface of the genetically replicable package; (ii) a second polypeptide segment that includes a cleavable peptide sequence; and (iii) a third polypeptide segment. It should be appreciated that the first and third peptide segments, both of which are desired, and both of which go into the final product, are initially joined together by the second polypeptide segment. They are separated from each other by a proteolytic agent that recognizes and cleaves the second polypeptide segment. The expressed single polypeptide may exist for a short period as a transitionary molecule. Alternatively, the cleavage may occur during the synthesis of the third polypeptide segment. This can avoid difficulties that may arise if the single, expressed polypeptide is toxic to the host organism in which it is expressed.

Preferably, a library of multimeric polypeptides is expressed by a population of genetically replicable packages to form a multimeric polypeptide display library. With respect to the genetically replicable package on which the variegated multimeric protein library is manifest, it will be appreciated that the replicable package will preferably have the ability to be (i) genetically altered to encode the multimeric polypeptide, (ii) maintained and amplified in culture, (iii) manipulated to display the multimeric protein product in a manner permitting the protein to interact with a target during an affinity separation step, and/or (iv) affinity-separated while retaining the nucleotide sequence encoding the multimeric polypeptide such that the nucleotide sequence of the multimeric polypeptide can be obtained.

Ideally, the display package includes a system that allows the sampling of very large variegated multimeric polypeptide display libraries, rapid sorting after each affinity separation round, and easy isolation of the multimeric polypeptide gene from purified display packages or further manipulation of that sequence. The most attractive candidates for this type of screening are prokaryotic organisms and viruses, as they can be amplified quickly, they are relatively easy to manipulate, and a large number of clones can be created.

Preferred genetic replication packages include, e.g. vegetative bacterial cells, bacterial spores, and most preferably, bacterial viruses. However, the present invention also contemplates the use of eukaryotic cells, including yeast and their spores, as potential genetic replication packages. The advantage of posttranslational modification and the possible harboring of structural complex proteins makes eukaryotic systems attractive for use in the instant invention. For a review of various eukaryotic systems, particularly the baculovirus expression system, for efficient display on the surface of virus particles as well as on the surface of virally infected cells, see Grabherr and Ernst (2001) Comb. Chem. High Throughput Screen, Apr;4(2):185-92, which is incorporated herein by reference. An advantage of the baculovirus system for peptide library screening is that expression of the multimeric polypeptides can be very high, e.g. greater than 1 million polypeptides/cell. A high expression level increases the likelihood of successful panning based on stoichiometry and/or contributes to polyvalent interactions with an immobilized target binding partner. Another advantage of the baculovirus system is that, similar to the phage display method, infectivity is exploited to amplify virus which is selected by the panning procedure. During the series of pannings, the DNA does not need to be isolated and used for subsequent transfections of cells.

An additional genetically replicable package contemplated by the present invention is the multimeric peptide on a plasmid, such as is described in U.S. Pat. No. 5,270,170, issued Dec. 14, 1993, which is incorporated by reference herein.

In addition to commercially available kits for generating phage display libraries, e.g., the Pharmacia Recombinant Phage Antibody System, catalog no. 27-9400-01; and the Stratagene SurfZAP™ phage display kit, catalog no. 240612, examples of methods and reagents particularly amenable for use in generating the variegated multimeric display library of the present invention can be found in, e.g., U.S. Pat. Nos. 5,223,409; 6,010,884; 5,863,765, and 5,948,635; Clackson et al. (1991) Nature 352:624-628; and Hoogenboom et al. (1991) Nuc. Acid Res. 19:4133-4137; each of which is incorporated herein by reference. Additional methods and reagents for use in the present invention include those described in U.S. Pat. Nos. 6,326,155; 5,837,500; 5,571,698; and 5,223,409; each of which is incorporated herein by reference. These systems can, with the modifications described herein, be adapted for use in the instant invention.

When the display is based on a bacterial cell, or a phage that is assembled periplasmically, the package will comprise at least two components. The first component is a secretion signal that directs the recombinant antibody to be localized on the extracellular side of the cell membrane of the package, or of the host cell when the genetic package is a phage. This secretion signal can be selected so as to be cleaved off by a signal peptidase to yield a processed, “mature” antibody. The second component is an anchoring peptide sequence for anchoring the multimeric polypeptide to the surface of the genetically replicable package. As described below, the anchoring peptide can be derived from a surface or coat protein native to the genetically replicable package.

When the package is a bacterial spore, or a phage whose protein coating is assembled intracellularly, a secretion signal directing the multimeric polypeptide to the inner membrane of the host cell is unnecessary. In these situations, the variegated multimeric polypeptide may include a derivative of a spore or phage coat protein amenable for use as a fusion protein.

Preferably, the multimeric polypeptide of the invention comprises an antibody, or fragment(s) thereof. The antibody component of the display preferably includes a V_{L} and C_{L} of a light chain, and the V_{H} and CH1 of a heavy chain, or portions thereof, of an antibody, e.g. cloned from B cells. It will be appreciated, however, that the antibody component may contain all or a portion of the V_{H} regions and/or the V_{L} regions without the addition of the constant regions, e.g. to generate an Fv fragment. Thus, typically, the display library will include the variable regions of both heavy and light chains to generate at least an Fv fragment. And preferably, at least a portion of the constant regions are included, e.g. to generate a Fab fragment. For clarity, some embodiments described herein detail the minimal antibody display as including the use of cloned light chain and heavy chain regions in a particular order to construct the fusion protein with the anchoring peptide. However, it should be readily understood that similar embodiments are possible in which the role of the light and heavy chains are reversed in the construction of the display library. Where the display antibody is to include more than two chains, two chains can be provided as a fusion protein with the genetically replicable package, and the other chain(s) can be provided as separate proteins on separate vectors, or alternatively, fused to the other two chains with additional cleavable linker sequences included such that the additional proteins are secreted and become associated with the fusion protein.

Either the light chain or the heavy chain, or both, may include a signal peptide leader sequence that will direct its secretion into the periplasm of the host cell. For example, several leader sequences have been shown to direct the secretion of antibody sequences in E. coli, such as OmpA (Hsiung et al. Bio/Technology (1986) 4:991-995), and (Better et al. Science 240:1041-1043), phoA (Skerra and Pluckthun, Science (1988) 240:1038).

In some embodiments of the invention, the heavy chain portion of the antibody display is derived from a library of different sequences, but the light chain is “fixed” (i.e., the same light chain for every antibody of the display), or vice versa. However, it will generally be preferred that the light chain is derived from a variegated light chain library, e.g., also cloned from the same population of B cells from which the heavy chain gene is cloned.

The number of possible combinations of heavy and light chains may exceed 10¹². To sample as many combinations as possible depends, in part, on the ability to recover large numbers of tranformants. For phage with plasmid-like forms, e.g., filamentous phage, electrotransformation provides an efficiency comparable to that of phage-transfection with in vitro packaging, in addition to a very high capacity for DNA input. This allows large amounts of vector DNA to be used to obtain very large numbers of transformants. The method described by Dower et al. (1988) Nucleic Acids Res., 16:6127-6145, for example, may be used to transform fd-tet derived recombinants at a rate of about 10⁷ transformants/μg of ligated vector into E. coli, and libraries may be constructed in fd-tet B1 of up to about 3×10⁸ members or more.

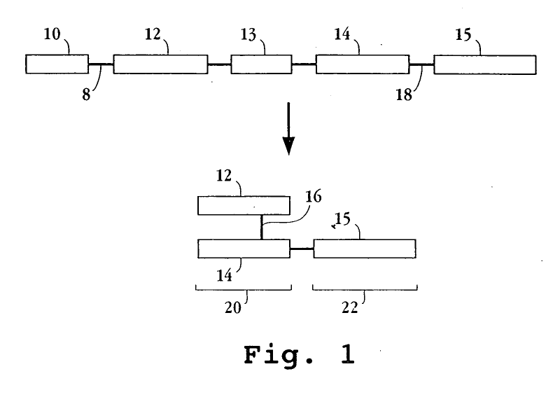

FIG. 1 illustrates an exemplary construction of a multimeric polypeptide, encoded by an expression vector having one or more vector segments, anchored onto a surface of a genetically replicable package used in practicing one embodiment of the invention. A vector segment encodes a polypeptide sequence that includes a first polypeptide segment 12. The vector segment also encodes a second polypeptide segment 13 that has a cleavable peptide sequence therein. A third polypeptide segment 14 having therein an anchoring peptide sequence 15 for anchoring the multimeric polypeptide to the surface of the genetically replicable package is also included. Optionally, a linker 18, which may be cleavable, links segment 14 to sequence 15, as shown.

In the embodiment shown in FIG. 1, a leader sequence 10 and is cleaved at point 8 by a signal peptidase prior to anchoring the multimeric polypeptide onto the surface of the replicable package. The fusion protein encoded by the fusion gene is shown before cleavage in segment 13 and after cleavage, illustrating dimeric polypeptide 20 assembly with the attached anchoring segment 22. Optionally, a covalent bond 16 links the first 12 and third 14 polypeptide segments.

Preferably, the anchoring peptide sequence 15 is a phage coat protein; the first polypeptide segment 12 is an antibody light chain; and the third polypeptide segment 14 is a variable domain and CH1 domain of a heavy chain segment. Thus, the dimeric polypeptide 20 is assembled as a Fab fragment anchored to a coat protein 22, e.g. gpIII or gpVIII of phage M13, as described in detail below. In this embodiment, the covalent bond 16 may be a disulfide bond that links the heavy and light chains together to form the Fab. Alternatively, the light and heavy chains may exchange positions in the fusion protein.

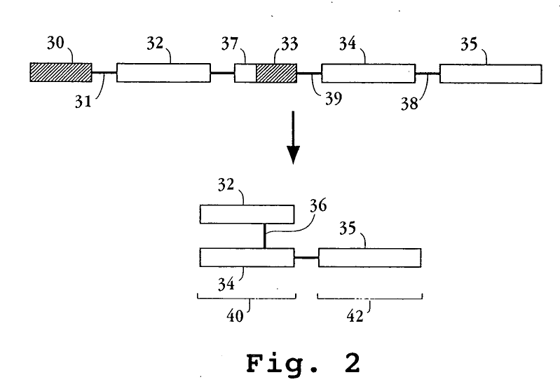

A related embodiment of the invention, as illustrated in FIG. 2, follows the same principles as above, except that the cleavable linker 37 is designed to be cleaved by a cytoplasmic protease (either endogenous or exogenous, as described in Section IA below), and an additional signal peptide 33 is included upstream of the third polypeptide 34. FIG. 2 shows the multimeric protein encoded by the fusion gene, before cleavage and after cleavage of the leader cleavage sites 31 and 39 and the linker cleavage site 37, translocation and dimeric assembly. Again, preferably, the anchoring peptide sequence 35 is a phage coat protein; the first polypeptide segment 32 is a light chain; and the third polypeptide segment 34 is the variable and CH1 domain of a heavy chain. Alternatively, the positions of the heavy and light chains are reversed. Thus, a dimeric polypeptide 40 is assembled as a Fab fragment anchored to a coat protein 42.

A. Cleavable Peptide Linker

As noted above, two polypeptide segments of the multimeric polypeptide are joined together by a peptide linker that has a cleavable peptide sequence. The cleavable peptide sequence is not found in either of the two polypeptide segments that it joins. In one embodiment, the cleavable peptide sequence is recognized as a protein cleavage site by a cleaving agent. In another embodiment, the cleavable peptide sequence is an autocleaving sequence derived from an intein. In yet another embodiment, the cleavable peptide sequence is an autocleaving sequence containing the sequence asp-pro, which cleaves under acidic conditions (Piszkiewicz et al [1970] Biochem. Biophys. Res. Commun. Vol. 40, pp. 1173-8).

Preferably, the cleaving agent is an enzyme, e.g. a proteolytic enzyme. The enzyme which carries out the cleavage could be an enzyme present in the host cytoplasm, periplasm or in a membrane, or elsewhere in the transformed organism, or an extracellular enzyme that has been produced by the organism. Alternatively, the enzyme could be added to the culture. Thus cleavage of the linking peptide may take place as the protein is being assembled in the periplasm, or in the surrounding culture medium.

This cleavage generally leads to a product in which at least one and possibly both of the polypeptide segments being linked are extended by a portion of the linking peptide, although the portion may be relatively small. Alternatively, the invention contemplates designing the linking peptide to be cut away completely by using two or more cleavage sites within the linker.

In one embodiment of the invention, cleavage of polypeptides may be achieved by chemical or enzymatic means. Thus, a protease enzyme may be used, such as trypsin, chymotrypsin, papain, gluc-C, endo lys-C, proteinase K, carboxypeptidase, calpain, subtilisin and pepsin. More preferably, the cleavable peptide sequence includes a sequence-specific cleavage site for cleavage of the peptide linker. The protease for cleavage may be urokinase, pro-urokinase, thrombin, enterokinase, plasmin, plasminogen, TGF-β, staphylokinase, thrombin, Factor IXa, Factor Xa, a metalloproteinase, an interstitial collagenase, a gelatinase, a stromelysin and/or any other protease known to those of skill in the art. Preferably, the cleavable peptide sequence is disordered and is cleavable by a protease that prefers disordered regions for cleavage. Exemplary proteases for use in the invention include degP, degQ, degS and/or tsp (Kolmar, H. et al. (1996) J. Bacteriology 178:5925-5929).

Alternatively, chemical agents such as cyanogen bromide can be used to effect cleavage. An exemplary cleavable sequence includes the sequence, from the N- to C-terminus, Asp-Pro, such that the sequence spontaneously cleaves in the presence of acid, e.g. pH 3-5.

In some embodiments of the invention, combinations of proteolytic agents may be preferred. The proteolytic agents can be immobilized in or on a support, or can be free in solution.

In one embodiment, the cleavable peptide sequence may include a self-cleaving domain derived from an intein. Inteins are also known as “protein introns,” “intervening protein sequences,” “protein spacers,” and the like. Inteins are somewhat analogous to introns found in mRNA molecules. As is the case for introns, inteins are spliced out of the respective polypeptide, resulting in joining of the portion of the polypeptide N-terminal to the intein (the “N-extein”) with the polypeptide portion that is to the C-terminal side of the intein (the “C-extein”). In one embodiment of the invention, however, the intein is spliced out of the polypeptide, without joining the adjacent polypeptide segments. Thus, the intein allows the separation of the desired polypeptide segments without the need for the production or supply of a protease. One advantage of this embodiment is that neither the genetically replicable package(s), e.g. phage, that may typically be sensitive to a protease, nor the desired polypeptide segments are compromised by exogenous or endogenous protease activity. Thus, the multimeric polypeptide(s) may be produced without reducing the viability of the genetically replicable package displaying the multimeric polypeptide. Exemplary self-cleaving intein mutant sequences may be found in U.S. Pat. No. 5,834,247, which is incorporated herein by reference.

The splicing reaction involves an acyl rearrangement between the S or O side chain of a cysteine, threonine or serine residue at the N-terminal of the intein with the peptide bond which connects the Cys, Thr or Ser residue to the N-extein. This rearrangement results in an intermediate in which the N-cysteine (or Ser or Thr) is attached to the adjacent extein by a thioester or ester, respectively. This intermediate then undergoes a trans-esterification reaction due to nucleophilic attack by an O or S-containing side chain of a Cys, Ser or Thr residue at the C-terminal end of the intein. This forms a branched polypeptide intermediate in which the N-extein is joined to a side chain of the Cys, Thr or Ser of the C-extein by a thioester or ester linkage. The intein is then released by cyclization of a conserved Asn residue at the carboxy end of the intein to form a succinimide derivative, followed by an O—N or S—N acyl shift and concomitant hydrolysis of the succinimide. The mechanisms of intein cleavage are discussed in, for example, Chong et al. (1998) Gene 192: 271-281; Evans et al. (1998) Protein Sci. 7: 2256-2264; and Paulus (1998) Chem. Soc. Reviews 27:375-386.

Inteins are described in, for example, U.S. Pat. Nos. 5,981,182, and 5,834,247, which are herein incorporate by reference in their entirety for all purposes and for the purpose of teaching inteins and intein chemistry. Inteins generally include amino acid residues that are conserved among inteins of different proteins. Intein motifs are described in, for example, Pietrokovski, S. (1994) Protein Science 3:2340-2350; Perler et al. (1997) Nuc. Acids Res. 25:1087-93; Pietrokovski, S. (1998) Protein Sci. 7:64-71. Other methods of identifying inteins are described in, for example, Dalgaard et al. (1997) J. Computational Biol. 4:193-214 and Gorbalenya, A. E. (1998) Nucleic Acids Res 26:1741-8. “INBASE” a compilation of known inteins by New England Biolabs, is found at http://circuit.neb.com/inteins/int id.html.

In some embodiments of the present invention, mutant inteins may be used in which only the amino-terminal end of the intein is capable of participating in the reaction. Such mutant inteins thus do not result in splicing of the N-extein to the C-extein. Instead, the N-extein is released from the intein upon attack by an activating compound that contains a nucleophilic group (e.g., a thiol or hydroxyl) under conditions conducive to intein cleavage. The activating compound then becomes attached to the end of the extein that was adjacent to the intein by a thioester or ester bond (see, e.g., Muir et al. (1998) Proc. Nat'l. Acad. Sci. USA 95: 6705-6710; Severinov and Muir (1998) J. Biol. Chem. 273: 16205-16209; Evans et al. (1998) Protein Sci. 7: 2256-2264). Suitable activating compounds that have nucleophilic groups include, for example, dithiothreitol (DTT), 2- mercaptoethanol, thiophenol, 2-mercaptoethanesulfonic acid, and cysteine-containing molecules, and the like. In some embodiments, the compounds contain 2-aminonucleophiles such as 2-aminothiols or 2-amino alcohols.

For some applications, the invention uses split inteins, in which the intein is split among two different polypeptide segments. The two molecules then undergo trans-splicing to excise the intein portions (termed the “n-intein” and the “c-intein”) and join the two exteins. An example of a naturally occurring intein occurs in the DnaE polypeptide of Synechocystis, as described in Wu et al. (1998) Proc. Nat'l. Acad. Sci. USA 95: 9226-9231 and Gorbalenya (1998) Nucl. Acids Res. 26: 1741-1748. Other trans-spliced inteins also occur naturally and are likewise suitable for use in the invention.

Because intein-mediated cleavage is somewhat dependent upon the amino acid present at the end of the adjacent polypeptide segment(s), the expression vector may also include one or more codons that add one or more amino acids which facilitate intein-mediated cleavage. Examples of suitable amino acids for cleavage are described in, for example, New England Biolabs catalog entitled “IMPACT[R]-CN” (Beverly, Mass.). The expression vector is then expressed, resulting in biosynthesis of the multimeric polypeptide. The polypeptide is subjected to the cleavage reactions discussed herein to release the desired segments of the multimeric polypeptide anchored on the surface of the genetically replicable package.

The invention is particularly well suited for the production of Fab fragments. The associative portions of the two chains will be their variable and constant domains or the binding regions thereof. The product will then be an Fab antibody fragment in which none, one or possibly both of the chains has a remnant of the linking peptide attached thereto. Because the peptide sequence which provides a link between the heavy chain domain and the light chain domain is cut after expression of the single polypeptide, there is greater freedom of choice in choosing the length of the linking peptide between them.

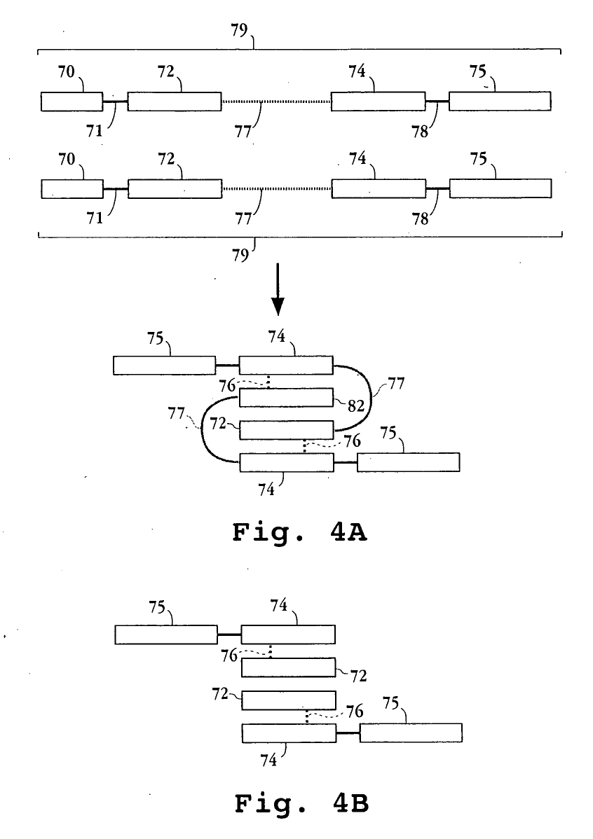

In one embodiment of the invention, the link between the antibody chains is sufficiently short, e.g. less than 10 amino acids, such that the two chains cannot associate together until the link is cut. The result of this may be that a folded monomeric single chain Fab is not produced as a transient product. This embodiment is schematized in FIGS. 4A and 4B, where the polypeptide segments dimerize to form a dimeric molecule with two potential binding sites. A vector segment encodes two polypeptide sequences 79, each of which include a first polypeptide segment 72, a second polypeptide segment 77, and a third polypeptide segment 74 having an anchoring peptide sequence 75 for anchoring the multimeric polypeptide to the surface of a genetically replicable package. Optionally, a linker 78, which may be cleavable as described herein, links third polypeptide segments 74 to the anchoring peptide sequence 75.

In one aspect of the invention, the second polypeptide segment is cleavable, and results in the multimeric polypeptide illustrated in FIG. 4B, where one, or preferably, both of the linkers 77 have been cleaved. Portions of the linkers may remain attached, or the linkers may be completely cleaved from the multimeric polypeptide as shown in FIG. 4B. In another embodiment, the second polypeptide segment remains uncleaved, as illustrated in FIG. 4A. Preferably, the polypeptide sequences are encoded by a single vector such that a dimeric molecule is formed. In one embodiment, not shown, one of the two anchoring peptides 75 are not formed, or are removed prior to dimerization.

In one embodiment, the first polypeptide segments 72 may be an antibody light chain or portion thereof, and the third polypeptide segments 74 are antibody heavy chains, or portions thereof. In another embodiment, this type of construct is used but the heavy and light chains exchange positions in the vector, i.e., the heavy chain precedes the light chain.

It should be appreciated that the above-described embodiments refering to polypeptides as first, second or third polypeptide segments may be oriented in either a N-terminal to C-terminal direction, or vice-versa. Thus, the first polypeptide segment may be at either the N-terminus or C-terminus of the polypeptide. Likewise, the third polypeptide, and/or the anchoring peptide segment, may be positioned at either the N-terminus or C-terminus of the polypeptide.

For some embodiments, the invention contemplates the use of amber (UAG), ocher (UAA) and/or opal (UGA) stop codons in the constructs immediately upstream of the phage coat protein-encoding nucleic acid sequence. In an amber suppressor background, this amber codon will sometimes insert an amino acid residue at the amber position, rather than reading it as a stop codon (Microbiology, Davis et al. Harper & Row, New York, 1980 pages 237, 245-47 and 274). The termination codon expressed in a wild type host cell results in the synthesis of the gene protein product without the phage coat attached. However, growth in a suppressor host cell results in the synthesis of detectable quantities of fused protein. Such suppressor host cells contain a tRNA modified to insert an amino acid in the termination codon position of the mRNA thereby resulting in production of detectible amounts of the fusion protein. Such suppressor host cells are well known and described, such as E. coli suppressor strain (Bullock et al. (1987) Bio Techniques 5, 376-379). Any acceptable method may be used to place such a termination codon into the nucleic acid encoding the multimeric polypeptide. Thus, in some fraction of time, the Fab dimers will have only one coat protein. This may be preferable for efficient attachment to the genetically replicable package, e.g., bacteriophage.

The suppressible codon may be inserted between the first gene encoding a polypeptide, and a second gene encoding at least a portion of a phage coat protein. Alternatively, the suppressible termination codon may be inserted adjacent to the fusion site by replacing the last amino acid triplet in the polypeptide or the first amino acid in the phage coat protein. When the phagemid containing the suppressible codon is grown in a suppressor host cell, it results in the detectable production of a fusion polypeptide containing the polypeptide and the coat protein. When the phagemid is grown in a non-suppressor host cell, the polypeptide is synthesized substantially without fusion to the phage coat protein due to termination at the inserted suppressible triplet encoding UAG, UAA, or UGA. In the non-suppressor cell the polypeptide is synthesized and secreted from the host cell due to the absence of the fused phage coat protein which otherwise anchored it to the genetically replicable package.

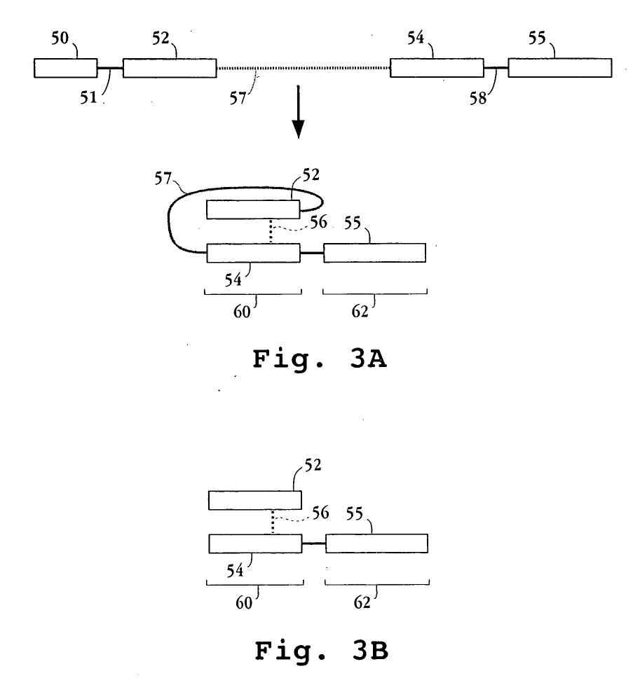

In another embodiment of the invention, as illustrated in FIG. 3A, the link 57 between the first polypeptide segment 52 and third polypeptide segment 54 is sufficiently long to form a single-chain Fab polypeptide 60, anchored to segment 62. Linker 57 is preferably cleaved as described herein and illustrated in FIG. 3B. Below the arrow in FIG. 3A, the processed and folded single-chain Fab fragment 60 is shown anchored to the phage coat protein 55. The dashed vertical line 56 represents the disulfide bond that covalently links the first and third polypeptide segments 52 and 54, respectively. Preferably, the first and third polypeptide segments are an antibody light and heavy chains. The anchoring peptide sequence 55 is preferably a phage coat protein, e.g. gpIII or gpVIII.

The nucleotide sequences encoding the three polypeptide segments of the multimeric polypeptides of the embodiments described above may be cloned in-frame into the vector using standard techniques of recombinant DNA technology.

B. Multimeric Polypeptides

The invention provides a method for identifying multimeric polypeptides which bind to molecules of interest, and vice versa. The multimeric polypeptides are produced from nucleotide libraries that encode peptides attached or anchored onto a surface. Preferably, the surface is a genetically replicable package as described in Section D, below. More preferably, the genetically replicable package is a bacteriophage, and the anchor is a bacteriophage structural protein. A method of affinity enrichment allows a very large library of multimeric polypeptides to be screened and the genetically replicable package carrying the desired multimeric polypeptide(s) selected. The nucleic acid may then be isolated from the genetically replicable package and the polypeptide segments of the library member sequenced, such that the amino acid sequence of the desired multimeric polypeptide is deduced therefrom. Using this method, a polypeptide identified has having a binding affinity for the desired molecule may then be produced or synthesized in bulk by conventional means.

By identifying the polypeptide de novo, one need not know the sequence nor structure of the multimeric polypeptide nor the characteristics of its binding partner. A significant advantage of the instant invention is that no prior information regarding an expected ligand structure is required to isolate ligands or molecules of interest. The multimeric polypeptide identified will thus have biological activity, which is meant to include at least a specific binding affinity for a selected molecule of interest, and in some instances will further include the ability to block the binding of other compounds, to stimulate or inhibit metabolic pathways, to act as a signal or messenger, and/or to stimulate or inhibit cellular activity.

As noted above, the multimeric polypeptide may be an antibody or a binding portion thereof. The antigen to which the antibody binds may be known and possibly sequenced, in which case the invention may be useful for mapping epitopes of the antigen. If the antigen is unknown, e.g., such as with certain autoimmune diseases, sera or other fluids from patients with the disease may be used to identify multimeric polypeptides, and consequently the antigen which elicits the autoimmune response. It is also within the scope of the present invention to tailor a multimeric polypeptide to fit a particular individual's disease. Once a polypeptide has been identified, it may itself serve as, or provide the basis for, the development of a vaccine, a therapeutic agent, and/or a diagnostic reagent.

The multimeric polypeptide may be a wide variety of substances in addition to antibodies. These include, e.g., growth factors, hormones, enzymes, interferons, interleukins, intracellular and intercellular messengers, lectins, cellular adhesion molecules and the like. See, e.g., U.S. Pat. No. 6,291,160, which is incorporated by reference herein. Ligands corresponding to these mulitmeric polypeptides can also be identified. Thus, although antibodies are widely available and conveniently manipulated, they are merely representative of the multimeric polypeptides of the present invention.

C. The Vector

The multimeric polypeptide, prepared according to the criteria as described herein, is encoded by nucleic acid segments that are inserted in an appropriate vector encoding three polypeptide segments. The vector is typically chosen to contain or is constructed to contain a cloning site located in the 5′ region of the gene encoding the anchoring peptide, so that the multimeric polypeptide is anchored or displayed such that it is accessible to binding partners in an affinity selection and enrichment procedure as described below.

An appropriate vector allows oriented cloning of the oligonucleotide sequences which encode the at least three polypeptide sequences—two of which form the multimeric polypeptide and one of which forms the cleavable linker sequence. In an exemplary vector of the present invention, the cloning region is located in the 5′ region of the gene encoding the bacteriophage structural protein such that the multimeric polypeptide is expressed at or within a distance of about 100 amino acid residues from the N-terminus of the mature coat protein. The coat protein is typically expressed as a preprotein, having a leader sequence. Thus, desirably, the polypeptide segments are inserted such that the N-terminus of the processed bacteriophage outer protein is the first residue of the multimeric polypeptide, i.e., between the 3′-terminus of the sequence encoding the leader protein and the 5′-terminus of the sequence encoding the mature protein or a portion of the 5′-terminus.

In one embodiment of the invention, a library is constructed by cloning a nucleic acid segment encoding the three polypeptides which include the cleavable linker sequence and antibody fragment library members, and any framework determinants into the selected cloning site. Using known recombinant DNA techniques (see generally, Sambrook et al., supra), a vector segment may be constructed which, inter alia, removes unwanted restriction sites and adds desired ones, reconstructs the correct portions of any sequences which have been removed (such as a correct signal peptidase site, for example), inserts framework residues, if any, and corrects the translation frame, if necessary, to produce active, infective phage. The central portion of the vector segment will generally contain two or more of the antibody domains and the cleavable linker sequence residues as described above. The sequences are ultimately expressed as peptides fused to or in the N-terminus of the mature coat protein on the outer, accessible surface of the assembled bacteriophage particles.

In another embodiment, the vector includes a sequence encoding a suppressor codon, such as TAG. In this embodiment, suppressor and nonsuppressor hosts may be utilized for production of the multimeric polypeptide with or without selected peptide regions under control of the suppressor host/vector system. Expression of other genes, such as those required for replication, packaging, and the like are not effected by the use of suppressor and nonsuppressor hosts.

The suppressor codon allows for the expression of the multimeric polypeptide described herein in a suitable suppressor host. In a nonsuppressor host, the suppressor codon allows for the translational termination of the upstream DNA translatable sequence. Preferably, a partially suppressor host is utilized such that a portion of the polypeptides are translationally terminated at a selected region, and another portion of the polypeptides are read-through. A preferred suppressor termination codon is either the amber or opal codons, and depends upon the suppressor strain to be utilized in conjunction with the vector or genetically replicable package, as is described herein. Suppressor and nonsuppressor hosts are described in U.S. application Ser. No. 2002/0910802, published Aug. 15, 2002, which is incorporated herein by reference.

D. Genetically Replicable Packages

As described above, one of the three polypeptide segments of the multimeric polypeptide includes an anchoring peptide for anchoring the multimeric polypeptide to the surface of a genetically replicable package. One of skill in the art will appreciate that a variety of genetically replicable packages may be employed in the present invention.

1. Phages as Genetically Replicable Packages

Bacteriophage are attractive prokaryotic-related organisms for use in the instant invention. Bacteriophage are excellent candidates for providing a display system of the variegated antibody library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass-screening programs, and they carry genetic information for their own synthesis within a small, simple package.

As the genes encoding the multimeric protein are inserted into the phage genome, the appropriate phage to be employed may be chosen to have one or more of the following properties: (i) the genome of the phage allows introduction of the heterologous genes either by tolerating additional genetic material or by having replaceable genetic material; (ii) the virion is capable of packaging the genome after accepting the insertion or subpackaging the genome after accepting the insertion or substitution of genetic material; and (iii) the display of the multimeric polypeptide on the phage surface does not disrupt virion structure sufficiently to interfere with phage propagation.

The morphogenetic pathway of the phage determines the environment in which the multimeric polypeptide will have the opportunity to fold. Periplasmically assembled phage are preferred as the multimeric polypeptide may contain essential disulfides. However, in certain embodiments in which the display package forms intracellularly, e.g. where λ phage are used, it has been demonstrated that disulfide-containing proteins have the ability to assume proper folding after the phage is released from the cell.

For a given bacteriophage, the preferred means for displaying the multimeric protein is with the use of a protein that is present on the phage surface, e.g. a coat protein. Filamentous phage can be described by a helical lattice; isometric phage, by an icosahedral lattice. Each monomer of each major coat protein ists on a lattice point and makes defined interactions with each of its neighbors. Proteins that fit into the lattice by making some, but not all, of the normal lattice contacts are likely to destabilize the virion by aborting formation of the virion as well as by leaving gaps in the virion so that the nucleic acid is not protected. Thus, in bacteriophage, unlike the cases of bacteria and spores, it is generally important to retain in the antibody fusion proteins those residues of hte coat protein that interact with other proteins in the virion. For example, when using the M13 cpVIII protein, the entire mature protein will generally be retained with the antibody fragment being added to the N-terminus of cpVIII, while on the other hand it can suffice to retain only the last 100 or fewer carboxy-terminal residues of the M13 cpIII coat protein in the multimeric protein fusion.

Under the appropriate induction, the multimeric protein library is expressed and exported, as part of the fusion protein, to the bacterial cytoplasm, such as when the λ phage is employed. The induction of the fusion protein(s) may be delayed until some replication of the phage genome, synthesis of some of the phage structural proteins, and assembly of some phage particles has occurred. The assembled protein chains then interact with the phage particles via the binding of the anchor protein on the outer surface of the phage particle. The cells are lysed and the phage bearing the library-encoded multimeric protein that corresponds to the specific library sequences carried in the DNA of that phage, are released and isolated from the bacterial debris.

To enrich and isolate phage that encode a selected multimeric polypeptide, and thus to ultimately isolate the nucleic acid sequences themselves, phage harvested from the bacterial debris are affinity-purified. As described below, when a multimeric polypeptide which specifically binds a particular target is desired, the target may be used ot retrieve phage displaying the desired multimeric polypeptide. The phage so obtained may then be amplified by infecting into host cells. Additional rounds of affinity enrichment followed by amplification may be employed until the desired level of enrichment is reached.

The enriched multimeric polypeptide/phage can also be screened with additional detection techniques such as expression plaque or colony lift. See, e.g. Young and Davis, Science (1983) 222:778-782, whereby a labeled target is used as a probe.

a. Filamentous Phage

Filamentous bacteriophages, which include M13, f1, f3, If1, Ike, Xf, Pf1, and Pf3, are a group of related viruses that infect bacteria. The F pili filamentous bacteriophage (Ff phage) infect only gram-negative bacteria by specifically adsorbing to the tip of F pili, and include fd, f1 and M13.

Compared to other bacteriophage, filamentous phage in general are attractive and M13 in particular has a number of advantages, including: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) it is easily cultured and stored, with no unusual or expensive media requirements for the infected cells, (viii) it has a high burst size, yielding 100 to 1000 M13 progeny per infected cell after infection; and (ix) it is easily harvested and concentrated.

The mature capsule of Ff phage is comprised of a coat of five phage-encoded gene products: cpVIII, the major coat protein product of gene VIII that forms the bulk of the capsule; and four minor coat proteins, cpIII and cpIV at one end of the capsule and cpVII and cpIX at the other end of the capsule. The length of the capsule is formed by 2500 to 3000 copies of cpVIII in an ordered helix array that forms the characteristic filament sturcture. The gene III-encoded protein (cpIII) is typically present in 4 to 6 copies at one end of the capsule and serves as the receptor for binding of the phage to its bacterial host in the initial phase of infection.

The phage particle assembly involves extrusion of the viral genome through the host cell's membrane. Prior to extrusion, the major coat protein cpVIII and the minor coat protein cpIII are synthesized and transported to the host cell's membrane. Both cpVIII and cpIII are anchored in the host cell membrane prior to their incorporation into the mature particle. In addition, the viral genome is produced and coated with cpV protein. During the extrusion process, cpV-coated genomic DNA is stripped of the cpV coat and simultaneously recoated with the mature coat proteins.

Both cpIII and cpVIII proteins include two domains that provide signals for assembly of the mature phage particle. The first domain is a secretion signal that directs the newly synthesized protein to the host cell membrane. The secretion signal is located at the amino terminus of the polypeptide and targets the polypeptide at least to the cell membrane. The second domain is a membrane anchor domain that provides signals for association with the host cell membrane and for association with the phage particle during assembly. The second signal for both cpVIII and cpIII includes a hydrophobic region for spanning the membrane.

The 50-amino acid mature gene VIII coat protein (cpVIII) is synthesized as a 73 amino acid precoat. cpVIII has been extensively studied as a model membrane protein because it can integrate into lipid bilayers such as the cell membrane in an asymmetric orientation with the acidic amino terminus toward the outside and the basic carboxy terminus toward the inside of the membrane. The first 23 amino acids constitute a typical signal-sequence that causes the nascent polypeptide to be inserted into the inner cell membrane. An E. coli signal peptidase (SP-I) recognizes amino acids 18, 21, and 23, and, to a lesser extent, residue 22, and cuts between residues 23 and 24 of the precoat. In one embodiment of the invention, this sequence is mutated to improve the display of the multimeric protein as described in Jestin, J L et al. (2001) Res. Microbiol., Mar;152(2):187-91. After removal of the signal sequence, the amino terminus of the mature coat is located on the periplasmic side of the innter membrane; the carboxy terminus is on the cytoplasmic side. About 3000 copies of the mature coat protein associate side-by-side in the inner membrane.

Mature gene VIII protein makes up the sheath around the circular ssDNA. The gene VIII protein can be a suitable anchor protein because its location and orientation in the virion are known. Preferably, the multimeric polypeptide is attached to the amino terminus of the mature M13 coat protein to generate the phage display library. As noted above, manipulation of the concentration of both the wild-type cpVIII and multimeric polypeptide/cpVIII fusion in an infected cell can be utilized to decrease the avidity of the display and thereby enhance the detection of high affinity antibodies directed to the target(s).

Another vehicle for displaying the multimeric polypeptide is by expressing it as a domain of a chimeric gene containing part or all of gene III, e.g., encoding cpIII. When monovalent displays are required, expressing the multimeric polypeptide as a fusion protein with cpIII is a preferred embodiment, as manipulation of the ratio of wild-type cpIII to chimeric cpIII during formation of the phage particles can be readily controlled. This gene encodes one of the minor coat proteins of M13. Genes VI, VII, and IX also encode minor coat proteins. Each of these minor proteins is present in about 5 copies per virion and is related to morphogenesis or infection. In contrast, the major coat protein is present in more than 2500 copies per virion. The gene VI, VII, and IX proteins are present at the ends of the virion; these three proteins are not posttranslationally processed. In particular, the single-stranded circular phage DNA associates with about five copies of the gene III protein and is then extruded through the patch of membrane-associated coat protein in such a way that DNA is encased in a helical sheath of protein.

The C-terminal cpIII 23-amino acid residue stretch of hydrophobic amino acids normally responsible for membrane anchor function can be altered in a variety of ways and retain the capacity to associate with membranes. Ff phage-based expression vectors were first described in which the cpIII amino acid residue sequence was modified by insertion of polypeptide targets or an amino acid residue sequence defining a single chain antibody domain (McCafferty et al. (1990), Science 348:552-554). It has been demonstrated that insertions into gene III may result in the production of novel protein domains on the virion outer surface (Smith (1985) Science 228:1315-1317; and de la Cruz et al. (1988) J. Biol. Chem. 263:4318-4322). Thus, the invention contemplates fusing the multimeric polypeptide to gene III at the site used by Smith and by de la Cruz et al., at a codon corresponding to another domain boundary or to a surface loop of the protein, or to the amino terminus of the mature protein.

Generally, the successful cloning strategy utilizing a phage coat protein, such as cpIII of filamentous phage fd, will provide expression of a multimeric polypeptide fused to the N-terminus of the coat protein and transport to the inner membrane of the host where the hydrophobic domain in the C-terminal region of the coat protein anchors the fusion protein in the membrane, with the N-terminus containing the multimeric polypeptide protruding into the periplasmic space.

Similar constructions are contemplated for other filamentous phage. Pf3 is a well known filamentous phage that infects Pseudomonos aerugenosa cells that harbor an IncP-I plasmid. The entire genome has been sequenced and the genetic signals involved in replication and assembly and protein interactions during its membrane protein insertion are known (Chen, M et al. (2002) J. Biol. Chem. 277(10):7670-5). The sequence has charged residues Asp-7, Arg-37, Lys-40, and Phe44 which are consistent with the amino terminus being exposed. Thus, to cause a multimeric polypeptide to appear on the surface of Pf3, a tripartite gene can be constructed which comprises a signal sequence known to cause secretion in P. aerugenosa, fused in-frame to gene fragments encoding a polypeptide sequence that includes a cleavable peptide sequence cleavable by a proteolytic agent, which is fused in-frame to a gene encoding the mature Pf3 coat protein, or fragment thereof. Optionally, DNA encoding a flexible linker of one to ten amino acids is introduced between the polypeptide sequence and the Pf3 coat protein gene. This tripartite gene is introduced into Pf3 so that it does not interfere with expression of any Pf3 genes. Once the signal sequence is cleaved off, the multimeric polypeptide is in the periplasm and the mature coat protein acts as an anchor and phage-assembly signal.

b. Bacteriophage φX174

The bacteriophage φX174 is a very small icosahedral virus that has been thoroughly studied by genetics, biochemistry, and electron microscopy (see Brussow H and Hendrix, R W (2002) Cell 108(1):13-6 for a comparative genomics review). Three gene products of ΦX174 are present on the outside of the mature virion: F (capsid), G (major spike protein, 60 copies per virion), and H (minor spike protein, 12 copies per virion). The G protein comprises 175 amino acids, while H comprises 328 amino acids. The F protein interacts with the single-stranded DNA of the virus. The proteins F, G, and H are translated from a single mRNA in the viral infected cells. As the virus is so tightly constrained because several of its genes overlap, φX174 is not typically used as a cloning vector because it can accept very little additional DNA. However, mutations in the viral G gene encoding the G protein can be rescued by a copy of the wild-type G gene carried on a plasmid that is expressed in the same host cell.

In one embodiment of the invention, one or more stop codons are introduced into the G gene such that no G protein is produced by the viral genome. The variegated multimeric polypeptide gene library can then be fused with the nucleic acid sequence of the H gene. An mount of the viral G gene equal to the size of multimeric polypeptide sequence is eliminated from the φX174 genome such that the size of the genome is not substantially changed. Thus, in host cells also transformed with a second plasmid expressing the wild-type G protein, the production of viral particles from the mutant virus is rescued by the exogenous G protein source. Where it is desirable that only one multimeric polypeptide be displayed per φX174 particle, the second plasmid can further include one or more copies of the wild-type H protein gene so that a mix of H and multimeric polypeptides/H proteins will be predominated by the wild-type H upon incorporation into phage particles.

c. Large DNA Phage

Phage such as λ or T4 have much larger genomes than do M13 or φX174, and have more complicated 3D capsid structures than M13 or φX174, with more coat proteins to choose from. In embodiments of the invention whereby the multimeric polypeptide library is processed and assembled into a functional form and associates with the bacteriophage particles within the cytoplasm of the host cell, bacteriophage λ and derivatives thereof are examples of suitable vectors. Variegated libraries expressing a population of functional antibodies have been generated in λ phage. See, e.g., Huse et al. (1989) Science 246:1275-81.

Bacteriophage T7 offers a combination of unique attributes that make it a preferable genetically replicable package. T7 is a double stranded DNA phage that has been studied extensively (Dunn, J. J. and Studier, F. W. (1983) J. Mol. Biol. 166:477-535; Steven, A. C. and Trus, B. L. (1986) Electron Microscopy of Proteins 5:1-35). Phage assembly takes place inside the E. coli cell and mature phage are released by cell lysis. In contrast to the assembly of filamentous phage, multimeric polypeptides displayed on the surface of T7 do not need to be capable of secretion through the cell membrane (Russel, M. (1991) Mol. Microbiol. 5:1607-1613). T7 has additional properties that make it an attractive genetically replicable package for use in the instant invention. It is very easy to grow and replicates more rapidly than either bacteriophage λ or filamentous phage. Plaques form within 3 hours at 37° C. and cultures lyse 1-2 hours after infection in liquid cultures, decreasing the time needed to perform the multiple rounds of growth usually required for successive rounds of selection. The T7 phage particle is extremely robust, and is stable, in harsh conditions that inactivate other phage.

In some embodiments of the invention, phage are introduced in a bacterial cell line that has a substantially oxidizing intracellular environment, e.g., the “Origami” strain as described in J. of Mol. Biol. (2002) vol. 315, pg. 1, which is incorporated by reference herein.

2. Bacterial Cells as Genetically Replicable Packages

Recombinant antibodies are able to cross bacterial membranes after the addition of appropriate secretion signal sequences to the N-terminus of the protein (Better et al. (1988) Science 240:1041-43; and Skerra et al. (1988) Science 240:1038-41). In addition, recombinant antibodies have been fused to outer membrane proteins for surface presentation. For example, one strategy for displaying antibodies on bacterial cells comprises generating a fusion protein by inserting the antibody into cell surface exposed portions of an integral outer membrane protein (Fuchs et al. (1991) Biotechnology 9:1370-72). In selecting a bacterial cell to serve as the genetically replicable package, any well-characterized bacterial strain will typically be suitable, provided the bacteria may be grown in culture, engineered to display the multimeric polypeptide library on its surface, and is compatible with the particular affinity selection process practiced in the instant method.

Among bacterial cells, preferred genetically replicable packages include Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized. See, e.g., Benz et al. (1988) Ann. Rev. Microbiol. 42:259-93; Balduyck et al. (1985) Biol. Chem. Hoppe-Seyler 366:9-14; Ehrmann et al. (1990) PNAS 87:7574-78; Heijne et al. (1990) Protein Engineering 4:109-12; Ladner et al. U.S. Pat. No. 5,223,409; Fuchs et al. (1991) Biotechnology 9:1370-72; and Goward et al. (1992) TIBS 18:136-40.

In one embodiment of the invention, the LamB protein of E. coli is used to generate a variegated library of multimeric polypeptides on the surface of a bacterial cell. See, e.g., Ronco et al. (1990) Biochemie 72:183-89. LamB of E. coli is a porin for maltose and maltodextrin transport, and serves as the receptor for adsorption of bacteriophages λ and Kb10. LamB is transported to the outer membrane if a functional N-terminal signal sequence is present. As with other cell surface proteins, LamB is synthesized with a typical signal-sequence that is subsequently removed. Thus, the variegated multimeric polypeptide gene library can be cloned into the LamB gene such that the resulting library of fusion proteins include a portion of LamB sufficient to anchor the protein to the cell membrane with the multimeric polypeptide oriented on the extracellular side of the membrane. Secretion of the extracellular portion of the fusion protein can be facilitated by inclusion of the LamB signal sequence, or other suitable signal sequence, as the N-terminus of the protein.

The E. coli LamB has also been expressed in functional form in S. typhimurium, V. cholerae, and K. pneumonia, so that one could display a population of multimeric polypeptides in any of these species as a fusion to E. coli LamB. Moreover, K. pneumonia expresses a maltoporin similar to LamB which could also be used in the instant invention. In P. aeruginosa, the D1 protein (a homologue of LamB) can be used. Similarly, other bacterial surface proteins such as PAL, OmpA, OmpC, OmpF, PhoE, pilin, BtuB, FepA, FhuA, Iuta, FecA and FhuE, may be used in place of LamB as a portion of the multimeric polypeptide in a bacterial cell.

3. Bacterial Spores as Genetically Replicable Packages

Bacterial spores also have desirable properties as genetically replicable packages in the instant invention. Spores are much more resistant than vegetative bacterial cells or phage to chemical and physical agents, and hence permit the use of a great variety of affinity selection conditions. Also, Bacillus spores neither actively metabolize nor alter the proteins on their surface.

Bacteria of the genus Bacillus form endospores which are extremely resistant to damage by heat, radiation, desiccation, and toxic chemicals (reviewed by Nicholson, W. L. (2002) Cell Mol. Life Sci. 59(3):410-6). This phenomenon is attributed to extensive intermolecular cross-linking of the coat proteins. In certain embodiments of the invention, such as those that include relatively harsh affinity separation steps, Bacillus spores can be the preferred genetically replicable package.

Viable spores that differ only slightly from wild-type are produced in B. subtilis even if any one of four coat proteins is missing. Moreover, plasmid DNA is commonly included in spores, and plasmid encoded proteins have been observed on the surface of Bacillus spores. Thus, it is possible during sporulation to express a gene encoding a chimeric coat protein that includes a multimeric polypeptide of the variegated gene library, without interfering materially with spore formation.

Several polypeptide components of B. subtilis spore coat have been characterized. The sequences of two complete coat proteins and amino-terminal fragments of two others have been determined. Fusion of the multimeric polypeptide sequence to cotC or cotD fragments is likely to cause the multimeric polypeptide to appear on the spore surface. The genes of each of these spore coat proteins are preferred as neither cotC or cotD are post-translationally modified (see Ladner et al., U.S. Pat. No. 5,223,409, which is incorporated herein by reference). 4. Selecting Multimeric Polypeptides

Upon expression, the variegated multimeric display may be subjected to affinity enrichment in order to select for multimeric polypeptides that bind preselected targets. The terms “affinity separation” or “affinity enrichment” includes, but is not limited to: (1) affinity chromatography utilizing immobilized targets, (2) immunoprecipitation using soluble targets, (3) fluorescence activated cell sorting, (4) agglutination, and (5) plaque lifts. The library of genetically replicable packages is ultimately separated based on the ability of the multimeric polypeptide to bind the target of interest.

Affinity chromatography includes a number of techniques that are known to those of skill in the art and can be adapted for use in the present invention. These include column chromatography, batch elution, ELISA and biopanning techniques. Typically, where the target is a component of a cell, rather than a whole cell, the target is immobilized on an insoluble carrier, such as sepharose or polyacrylamide beads, or, alternatively, the wells of a microtitre plate. As described below, in instances where no purified source of the target is readily available, such as the case with many cell surface receptors, the cells on which the target is displayed may serve as the insoluble matrix carrier.

The population of genetically replicable packages may be applied to the affinity matrix under conditions compatible with the binding of the multimeric polypeptide to a target. The population is then fractionated by washing with a solute that does not greatly effect specific binding of multimeric polypeptides to the target, but which substantially disrupts any non-specific binding of the package to the target or matrix. A certain degree of control can be exerted over the binding characteristics of the multimeric polypeptides recovered from the display library by adjusting the conditions of the binding incubation and subsequent washing. The temperature, pH, ionic strength, divalent cation concentration, and the volume and duration of the washing can select for multimeric polypeptides within a particular range of affinity and specificity.

Selection based on slow dissociation rate, which is usually predictive of high affinity, is a very practical route. This may be accomplished by increasing the volume, number, and/or length of the washes. In each case, the rebinding of dissociated multimeric polypeptide/package is prevented, and with increasing time, multimeric polypeptide/packages of higher and higher affinity are recovered. Moreover, additional modifications fo the binding and washing procedures may be applied to find multimeric polypeptides with special characteristics. The affinities of some multimeric polypeptides, e.g., antibodies, are dependent on ionic strength or cation concentration. This is a useful characteristic for antibodies to be used in affinity purification of various proteins when gentle conditions for removing the protein from the antibody are required. Specific examples are antibodies which depend on Ca⁺⁺ for binding activity and which lose or gain binding affinity in the presence of EGTA or other metal chelating agent. Such antibodies may be identified in the recombinant antibody library by a double screening technique isolating first those that bind the target in the presence of Ca⁺⁺, and by subsequently identifying those in this group that fail to bind in the presence of EGTA.

When desired, after “washing” to remove non-specifically bound genetically replicable packages, specifically bound packages may be eluted by either specific desorption, e.g. using excess target, or non-specific desorption, e.g. using pH, polarity reducing agents, or chaotropic agents. In preferred embodiments, the elution protocol does not kill the organism used as the genetically replicable package such that the enriched population of display packages can be further amplified by reproduction. Eluants include salts, acid, heat, and soluble forms of the target. Neutral solutes, such as ethanol, acetone, ether, and urea are other examples of reagents useful for eluting the bound genetically replicable packages.

Preferably, affinity enriched genetically replicable packages are iteratively amplified and subjected to further rounds of affinity separation until enrichment of the desired binding activity is detected. Specifically bound genetically replicable packages, particularly bacterial cells, may not need to be eluted, but rather the matrix-bound packages can be used directly to inoculate a suitable growth media for amplification.

In one embodiment of the invention, the multimeric polypeptide can be formed on the surface of the display package such that it is susceptible to proteolytic cleavage that severs the covalent linkage of at least the target binding sites of the displayed multimeric polypeptide from the remaining package. For example, where the cpIII coat protein of M13 is employed, such a strategy can be used to obtain infectious phage by treatment with an enzyme that cleaves between the multimeric polypeptide portion and cpIII portion of a tail fiber fusion protein, e.g., by using an enterokinase cleavage recognition sequence.

DNA prepared from eluted phage may be transformed into host cells by electroporation or other well known chemical means to further minimize ay problems associated with defective infectivity. The cells are cultivated for a period of time sufficient for marker expression, and selection is applied as typically performed for DNA tranformation. The colonies are amplified, and phage harvested for a subsequence round or rounds of panning.

The multimeric polypeptides of each of the genetically replicable packages can be tested for biological activity, e.g. a desired binding specificity, either prior to, or after, isolation of the packages that encode the multimeric polypeptides.

E. Generation of Multimeric Polypeptide Libraries

The variegated multimeric polypeptide libraries of the invention may be generated by any of a number of methods. In an exemplary embodiment, following application of an immunization step, an antibody repertoire of a resulting B-cell pool is cloned. Methods for obtaining the DNA sequence of the variable regions of a diverse population of immunoglobulin molecules are well known in the art, e.g., by using a mixture of oligomer primers and PCR. For example, mixed oligonucleotide primers corresponding to the 5′ leader sequences and/or framework sequences, as well as primers to a conserved 3′ constant region can be used for PCR amplification of the heavy and light chain regions from a number of antibodies. Additional techniques for generating antibodies and antibody fragments are reviewed in Tse, E et al. (2002) Methods Mol. Biol. 185:433-46. Oligonucleotide primers may be unique, degenerate, and/or incorporate inosine at degenerate positions. Restriction endonuclease recognition sequences may also be incorporated into the primers to allow for the cloning of the amplified fragment into a vector in a predetermined direction and/or reading frame for expression.

F. Utility

The invention may be used in a broad range of applications, including for the selection of multimeric polypeptides having effects on proliferation, differentiation, cell death, and/or cell migration. In one embodiment of the invention, multimeric polypeptides, e.g. antibodies, that have antiproliferative activity with respect to one or more types of cells may be identified. For example, the multimeric polypeptide library can be panned with target cells for which an antiproliferative is desired in order to enrich for antibodies that bind to that cell. The multimeric polypeptide library may also be panned against one or more control cell lines in order to remove multimeric polypeptides that bind the control cells. Thus, the multimeric polypeptide library is then tested and enriched for multimeric polypeptides that selectively bind the target cell relative to the control cells. Thus, for example, an antibody library enriched for antibodies that preferentially bind tumor cells relative to normal cells, preferentially bind p53-cells relative to p53+ cells, or exhibit any other differential binding characteristic may be selected.

III. Libraries

As discussed above, another aspect of the invention provides libraries and vectors for practice of the methods described herein. The libraries may be monovalent or polyvalent libraries, including diabody libraries and preferably are Fab libraries expressed by phage.

The libraries may take a number of forms. Thus, in one embodiment the library is a collection of cells containing members of the phage display library, while in another embodiment, the library comprises a collection of isolated phage, and in still another embodiment, the library includes nucleic acids encoding a phage display library. The nucleic acid molecules may be phagemid vectors encoding the antibody fragments and ready for subcloning into a phage vector or the nucleic acid molecules may be a collection of phagemid already carrying the subcloned antibody fragment-encoding nucleic acids.

Another embodiment of the invention is directed to a method for creating a library of receptor proteins or any proteins which show variability. Receptor proteins which may be utilized in this method may be any eukaryotic or prokaryotic proteins which have variable regions including T-cell receptors such as the TcR, B-cell receptors including immunoglobulins, natural killer cell (NK) receptors, macrophage receptors and portions and combinations thereof. Briefly, a sample of biological tissue, such as normal tissue, neoplastic tissue, infected tissue, tissues containing extracellular matrix (ECM) proteins, or any abnormal tissue, is introduced to a cell population capable of producing the receptor proteins. The cell population is fixed and the cells permeabilized. The variable region mRNAs of the receptor proteins are reverse transcribed into cDNA sequences using a reverse transcriptase. The cDNA sequences are PCR amplified and linked with a proteolytically cleavable linker as described above, preferably by hybridization of complementary sequences at the terminal regions of these cDNAs. The linked sequences are PCR amplified to create a population of DNA fragments which encode the variable regions with or without any portion of any constant regions of the receptor proteins. These DNA fragments contain the variable regions linked with a proteolytically cleavable linker, and are cloned in-mass into expression vectors. Useful expression vectors are described in section II.C., above, and include phages such as display phages, cosmids, viral vectors, phagemids or combinations thereof. The vectors are transformed into host organisms and the different populations of organisms expanded. The expression vectors which encode the recombinant receptor proteins are selected and the subpopulation expanded. The sub-population may be subcloned into expression vectors, if necessary, which contain receptor constant region genes in-frame and the library again expanded and expressed to produce the sub-library of selected receptor proteins. Chimeric libraries can be easily created by cloning the selected variable region genes into expression vectors containing constant region genes of other proteins such as antibody constant region genes or T cell receptor genes. The selected sub-libraries can be used directly or transferred to other expression vectors before transfection into host cells. Host cells may be T cells derived from the patient which, when introduced back into the patient, express the receptor library on their surface. This type of T cell therapy can be used to stimulate an immune response to treat a number of diseases as described herein.

Using the methods discussed above for the creation of antibody libraries and libraries of T cell receptors, libraries of chimeric fusion proteins can be created which contain the variable regions of antibodies joined with the constant regions of T cell receptor. Such libraries may be useful for treating or preventing diseases and disorders, as described above, by stimulating or enhancing a patient's immune response. For example, antigen binding to the T cell receptor is an integral part of the immune response. By providing a chimeric antibody/TcR protein library and by transfecting this library into a patient population of T cells, the patient's own immune response may be enhanced to fight off a disease or disorder that it could not otherwise successfully overcome.

IV. Kits

Another aspect of the invention provides kits for practice of the methods described herein. The kits preferably include members of a phage display library, e.g., phage particles, vectors, and/or cells containing phage. The assay kits may additionally include any of the other components described herein for the practice of methods or assays of the invention. Such materials include, but are not limited to, helper phage, or or more bacterial or eukaryotic cell lines, buffers, antibiotics, labels, and the like.

In addition, the kits may optionally include instructional materials containing directions or protocols disclosing the methods described herein. While the instructional materials typically comprise written or printed materials, they are not limited to such. Any medium capable of storing such instructions and communicating them to an end user is contemplated by this invention. Such media include, but are not limited to electronic storage media, e.g., magnetic discs, tapes, cartridges, chips, and/or optical media such as CD ROMS, and the like. Such media may include addresses to internet sites that provide such instructional materials.

One embodiment of the invention is directed to a diagnostic kit for the detection of a disease or disorder in a patient, or a contaminant in the environment comprising a library of antigen-, tissue- or patient-specific antibodies or antibody fragments.

The diagnostic kit can be used to detect diseases such as bacterial, viral, parasitic or mycotic infections, neoplasias, or genetic defects or deficiencies. The biological sample may be blood, urine, bile, cerebrospinal fluid, lymph fluid, amniotic fluid or peritoneal fluid, preferably obtained from a human. Libraries prepared from sample obtained from the environment may be used to detect contaminants in samples collected from rivers and streams, salt or fresh water bodies, soil or rock, or samples of biomass. The antibody may be a whole antibody such as an IgG or, preferably, an antibody fragment such as an Fab fragment. The library may be labeled with a detectable label or the kit may further comprise a labeled secondary antibody that recognizes and binds to antigen-antibody complexes. Preferably, the detectable label is visually detectable such as an enzyme, fluorescent chemical, luminescent chemical or chromatic chemical, which would facilitate determination of test results for the user or practitioner. Additional components of such kits may be found in U.S. Pat. No. 6,335,163, issued Jan. 1, 2002, which is incorporated by reference herein in its entirety.

The kits may further comprise agents to increase stability, shelf-life, inhibit or prevent product contamination and/or increase detection rates. Useful stabilizing agents include water, saline, alcohol, glycols including polyethylene glycol, oil, polysaccharides, salts, glycerol, stabilizers, emulsifiers and combinations thereof. Useful antibacterial agents include antibiotics, bacterial-static and bacterial-toxic chemicals. Agents to optimize speed of detection may increase reaction speed such as salts and buffers.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE I
Sequences
Sequences of the Invention

| SEQ ID NO: | Sequence |
|---|---|
| 1 | Asp-Pro |

1. An expression vector for expressing a multimeric polypeptide anchored on a surface of a genetically replicable package formed by a host, the expression vector comprising:
a vector segment encoding a polypeptide sequence having;
i. a first polypeptide segment,
ii. a second polypeptide segment having therein a cleavable peptide sequence cleavable by a proteolytic agent, and,
iii. a third polypeptide segment having therein an anchoring peptide sequence for anchoring said multimeric polypeptide to said surface of said genetically replicable package,
the second polypeptide segment being between the first polypeptide segment and the third segment,
whereby the cleavable peptide sequence is cleaved by the proteolytic agent and whereby the first segment associates with the third segment to form the multimeric polypeptide.

2. The expression vector of claim 1, wherein the first and third polypeptide segments comprise an amino acid sequence derived from antibody light and heavy chains.

3. The expression vector of claim 1, wherein the first and third polypeptide segments comprise the antigen binding regions of the variable domains of antibody light and heavy chains.

4. The expression vector of claim 1, wherein the first polypeptide segment comprises the variable domain and the constant domain of an antibody light chain, and the third polypeptide segment comprises the variable domain and a constant domain of the antibody heavy chain, such that when the first and third segments associate, the product is a Fab antibody fragment.

5. The expression vector of claim 1, wherein the first polypeptide segment comprises the variable domain and the CH1 domain of an antibody heavy chain, and the third polypeptide segment comprises the variable domain and the constant domain of the antibody light chain, such that when the first and third segments associate, the product is a Fab antibody fragment.

6. The expression vector of claim 1, wherein the first polypeptide segment comprises the variable domain and the constant domain of the antibody light chain, and the third polypeptide segment comprises the variable domain and the CH1 domain of an antibody heavy chain, such that when the first and third segments associate, the product is a Fab antibody fragment.

7. The expression vector of claim 1, wherein the first and third polypeptide segments comprise the variable domains of the light and heavy chains of a single antibody such that when the first and third segments associate, the product is an Fv antibody fragment.

8. The expression vector of claim 1, wherein the first polypeptide segment is N-terminal to the second polypeptide segment, and wherein the second polypeptide segment is N-terminal to the third polypeptide segment, and wherein the vector segment encoding the third polypeptide segment further includes one or more suppressable nonsense codon(s) N-terminal to the anchoring segment.

9. The expression vector of claim 1, wherein the third polypeptide segment further includes a cleavable peptide sequence cleavable by a second proteolytic agent.

10. The expression vector of claim 9, wherein the first and second proteolytic agents are identical.

11. The expression vector of claim 1, wherein the proteolytic agent is selected from the group consisting of a chemical proteolytic agent and an enzymatic proteolytic agent.

12. The expression vector of claim 1, wherein the proteolytic agent is expressed by the host.

13. The expression vector of claim 1, wherein the proteolytic agent is added such that it contacts and cleaves the second polypeptide segment.

14. The expression vector of claim 11, wherein the chemical proteolytic agent is an acid.

15. The expression vector of claim 1, wherein the cleavable peptide sequence comprises the sequence represented by SEQ ID NO:1.

16. The expression vector of claim 1, wherein the cleavable peptide sequence is not found in either the first or third polypeptide segments, and is recognized as a protein cleavage site by a proteolytic agent encountered in the host.

17. The expression vector of claim 1, wherein the polypeptide sequence further comprises one or more leader sequence(s) positioned upstream of the first polypeptide segment or third polypeptide segment or both first and third polypeptide segments.

18. The expression vector of claim 1, wherein the anchoring peptide comprises a segment encoding a phage coat protein.

19. The expression vector of claim 1, wherein the expression vector is selected from the group consisting of plasmids, phages, cosmids, phagemids, and viral vectors.

20. The expression vector of claim 1, wherein the expression vector is selected from the group consisting of M13, f1, fd, If1, Ike, Xf, Pf1, Pf3, λ, T4, T7, P2, P4, φX-174, MS2 and f2.

21. The expression vector of claim 1, wherein the genetically replicable package is selected from the group consisting of a bacteriophage, a virus, a cell and a spore.

22. The expression vector of claim 21, wherein the cell is a bacterial cell.

23. The expression vector of claim 22, wherein the bacterial cell is selected from the group consisting of strains of Escherichia coli, Salmonella typhimurium, Pseudomonas aeruginosa, Klebsiella pneumonial, Neisseria gonorrhoeae, and Bacillus subtilis.

24. The expression vector of claim 21, wherein the cell is a yeast cell.

25. The expression vector of claim 1, wherein the genetically replicable package is a filamentous bacteriophage specific for Escherichia coli and the anchoring peptide is a phage coat protein selected from the group consisting of coat protein III, coat protein pVI and coat protein VIII.

26. The expression vector of claim 25, wherein the filamentous bacteriophage is selected from the group consisting of M13 and fd.

27. The expression vector of claim 1, wherein the proteolytic agent is encoded by a nucleic acid sequence in the expression vector.

28. The expression vector of claim 1, wherein the proteolytic agent is encoded by a nucleic acid sequence in a second expression vector.

29. The expression vector of claim 1, wherein the cleavable peptide sequence comprises a disordered region cleavable by the proteolytic agent.

30. The expression vector of claim 1, wherein the cleavable peptide sequence comprises a specific peptide cleavage site cleavable by the proteolytic agent.

31. The expression vector of claim 1, wherein the cleavable peptide sequence includes a cleavage site for urokinase, pro-urokinase, thrombin, enterokinase, plasmin, plasminogen, TGF-β, staphylokinase, thrombin, Factor IXa, Factor Xa, a metalloproteinase, an interstitial collagenase, a gelatinase or a stromelysin.

32. The expression vector of claim 1, wherein the cleavable peptide sequence is cleavable by a protease selected from the group consisting of degP, degQ, degS and tsp.

33. The expression vector of claim 1, wherein the cleavable peptide sequence comprises a self-cleaving domain.

34. The expression vector of claim 33, wherein the self-cleaving domain is derived from an intein.

35. A host cell comprising the expression vector of claim 1.

36. The host cell of claim 35, wherein the proteolytic agent is a native proteolytic agent.

37. The host cell of claim 35, wherein the proteolytic agent is localized in the periplasm.

38. The host cell of claim 35, wherein the proteolytic agent is localized in the cytoplasm.

39. A method of producing a multi-subunit protein, comprising
transforming a host cell with the expression vector of claim 1, and
displaying the multi-subunit protein encoded by the vector onto the surface of the genetically replicable package.

40. The method of claim 39, wherein the vector comprises nucleotide sequences encoding functional portions of heterodimeric receptors selected from the group consisting of antibodies, T cell receptors, integrins, hormone receptors and transmitter receptors.

41-81. (canceled)


SEQUENCE LISTING (1 sequence)

SEQ ID NO: 1 - protein, 2 aa, Artificial Sequence
  cleavable peptide sequence
DP